(19) United States
(12) Patent Application Publication
(10) Pub. No.: US 2013/0131378 A1
(43) Pub. Date: May 23, 2013

(54) PROCESS FOR PREPARING 4-NITRO-OXY-METHYL-BENZOIC ACID

(71) Applicants: Luis Anglada, Barcelona (ES); Albert Palomer, Barcelona (ES); Luis Sobral, Loures (PT)

(72) Inventors: Luis Anglada, Barcelona (ES); Albert Palomer, Barcelona (ES); Luis Sobral, Loures (PT)

(73) Assignees: NICOX S.A., SOPHIA ANTIPOLIS-VALBONNE (FR); FERRER INTERNACIONAL, S.A, BARCELONA (ES)

(21) Appl. No.: 13/509,906

(22) PCT Filed: Nov. 15, 2010
(86) PCT No.: PCT/EP2010/067444

§ 371 Date: Jun. 8, 2012

(30) Foreign Application Priority Data
Nov. 16, 2009 (ES) P 200931000

(51) Int. Cl. C07C 51/347

(52) U.S. Cl. CPC C07C 51/347
USPC 562/473

(57) ABSTRACT

This invention relates to a new process for preparing 4-nitro-oxy-methyl-benzoic acid, comprising the following steps: a) reaction of 4-chloromethyl-benzoic acid with silver nitrate and in the presence of an acid as a catalyst in acetonitrile at reflux temperature, followed by cooling and adding of a polar aprotic solvent; b) separation of the silver salts by filtration, followed by washout with a polar aprotic solvent; c) precipitation of the 4-nitro-oxy-methyl-benzoic acid with water from the filtrate obtained in step b); and d) drying of the 4-nitro-oxy-methyl-benzoic acid.

### FIELD OF THE INVENTION

This invention relates to a new process for preparing 4-nitro-oxy-methyl-benzoic acid, a compound used as an intermediate product in the manufacture of pharmaceutical substances, specifically for steroidal anti-inflammatory drugs.

### BACKGROUND ART

The preparation of 4-nitro-oxy-methyl-benzoic acid, with formula (I), has been previously described in the literature by several authors (1-10) from a 4-(bromo or chloro)-methyl-benzoic acid (II, X=Br, Cl) by treatment with silver nitrates in an acetonitrile solution or in solution within a mixture of tetrahydrofurane (THF) and acetonitrile. Depending on the experimental conditions the reported yields range from 54 to 84% (Table 1).

TABLE 1

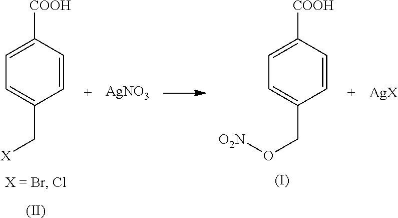

| Reagent (II) | Solvent | Reaction conditions | Yield | Ref. |
|---|---|---|---|---|
| X = Br | CH₃CN | RT overnight | 84% | (1) |
| X = Br | CH₃CN | RT 24 h | 83% | (2) |
| X = Br | CH₃CN | reflux 8 h + RT 16 h | 80% | (3) |
| X = Br | CH₃CN | reflux 8 h + RT 16 h | 80% | (4) |
| X = Br | THF/CH₃CN | RT overnight + 50° C. 1 h | 73% | (5) |
| X = Cl | CH₃CN | RT 2 h | 54% | (6) |
| X = Br | CH₃CN | reflux 8 h + RT 16 h | 80% | (7) |
| X = Br | THF/CH₃CN | RT overnight + 50° C. 1 h | 73% | (8) |
| X = Cl | CH₃CN | RT 2 h | 54% | (9) |
| X = Br | CH₃CN | RT 12 h | 79% | (10) |

(1) Endres S. et al., European Journal of Medicinal Chemistry (1999), 34(11), 895-901
(2) Wessler C. et al., European Journal of Medicinal Chemistry (2003), 38(6), 581-586
(3) Scaramuzzino G., EP1336602A1, Pub. 20030820
(4) Scaramuzzino G., WO03094923A1, Pub. 20031120
(5) Earl R. A. et al., WO04004648A2, Pub. 20040115
(6) Breschi M. C. et al., Journal of Medicinal Chemistry (2006), 49(8), 2628-2639
(7) Scaramuzzino G., IT 2002MI0402A1, Pub. 20030828
(8) Wey S. J. et al., Journal of Medicinal Chemistry (2007), 50(25), 6367-6382
(9) Calderone V. et al., Journal of Pharmacy and Pharmacology (2008), 60(2), 189-195
(10) Chong W. et al., WO08075152A1, Pub. 20080626

Similarly, the production of (I) has been described by nitration of (II, X=OH) with nitric acid and acetic anhydride (11) at low temperature, from −30° C. -10° C., the yield being of 83% (Table 2).

TABLE 2

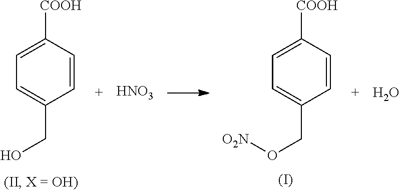

| Reagent (II) | Solvent | Reaction conditions | Yield | Ref. |
|---|---|---|---|---|
| X = OH | (CH₃CO)2O | −30° C. 15 min + −10° C. 2 h | 83% | (11) |

(11) McIntyre D. G., US6696592B2, Pub. 20040224

The processes shown in Table 1 are usually preferable due to the lower aggressiveness of the solvents and the easier reaction conditions. Also, the starting product with the greatest ease of handling, due to its greater stability and less unpleasant organoleptic effects, especially with views to industrialising the process, is 4-chloromethyl-benzoic acid (III) (II, X=Cl).

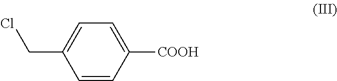 [SMILES: O=C(O)c1ccc(CCl)cc1]

However, the use of this starting product presents two important problems, which are its low yield (54%) and the formation of the dimer with formula (IV).

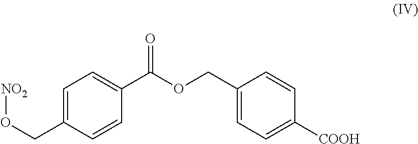 [SMILES: O=C(O)c1ccc(COC(=O)c2ccc(CO[N+](=O)[O-])cc2)cc1]

The presence of (IV) is an obstacle in the in subsequent synthesis of the steroidal anti-inflammatory drug (V), a compound described in WO2007025632A2.

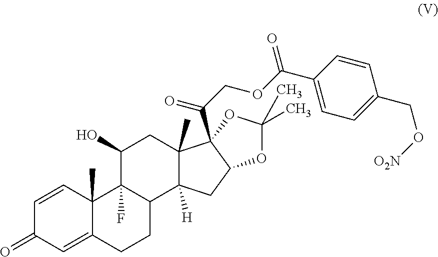

It is therefore necessary to achieve a process for obtaining (I) with a good yield and with the least presence of impurity (IV).

The authors of the present invention have achieved a new industrial process for obtaining (I) that leads to the product with a much greater yield and greater purity.

### SUMMARY OF THE INVENTION

In a single aspect, the invention provides a new industrial process for preparing 4-nitro-oxy-methyl-benzoic acid with an excellent yield and greater purity.

### DETAILED DESCRIPTION OF THE INVENTION

This invention has as an object to provide a process for preparing 4-nitro-oxy-methyl-benzoic acid (I) that is based on the known reaction between 4-chloromethyl-benzoic acid (III) and silver nitrate. However, the applicants have discovered that the presence of an acid as a catalyst leads to the production of (I) with a great yield and with a proportion of impurity (IV) much below that obtained without said catalyst.

Indeed, during preliminary experiments in which solvents and reaction conditions were varied and different catalysts were tested, the applicants found that, despite the possibility of obtaining substantially greater yields than those described in the literature, the maximum purity of 4-nitro-oxy-methyl-benzoic acid (I) obtained by reaction with 4-chloromethyl-benzoic acid (III) with silver nitrate was at the most of 98.74% (HPLC) and that the presence of the by-product (IV) could not be reduced further than 0.82% (HPLC), which is an excessive proportion since this impurity produces in turn other by-products that are very difficult to eliminate in the subsequent manufacture of the steroid (V).

The process for preparing 4-nitro-oxy-methyl-benzoic acid (I), which constitutes the single aspect of the invention, comprises the following steps:
a) reaction of 4-chloromethyl-benzoic acid (III)

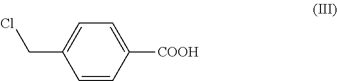 [SMILES: O=C(O)c1ccc(CCl)cc1]

with silver nitrate and in the presence of an acid as a catalyst in acetonitrile at reflux temperature, followed by cooling and adding of a polar aprotic solvent;
b) separation of the silver salts by filtration, followed by washout with a polar aprotic solvent;
c) precipitation of compound (I) with water from the filtrate of step b); and
d) drying of the compound (I).

In a preferred embodiment, the acid is chosen from the group consisting of benzene sulphonic, hydrobromic, hydrochloric, chloroacetic, chloro sulphonic, ethane sulphonic, phosphoric, methane sulphonic, nitric, p-chloro benzene sulphonic, p-toluene sulphonic, sulphuric, trichloroacetic, trichloromethane sulphonic, trifluoroacetic and trifluoromethane sulphonic and the like and mixtures thereof. The acid chosen is preferably sulphuric acid.

In a preferred embodiment, the polar aprotic solvent in step a) is chosen from the group consisting of acetonitrile, benzonitrile, dimethylformamide, dimethyl sulphoxide, dioxane, N-methyl-2-pyrrolidone, propionitrile and tetrahydrofurane and the like and mixtures thereof. Said solvent is preferably dimethylformamide.

In another preferred embodiment, the polar aprotic solvent in step b) is chosen from the group consisting of acetonitrile, benzonitrile, dimethylformamide, dimethyl sulphoxide, dioxane, N-methyl-2-pyrrolidone, propionitrile and tetrahydrofurane and the like and mixtures thereof. Said solvent is preferably dimethylformamide.

In another preferred embodiment, step c) comprises a subsequent washout with (C₁-C₃)alkanol. Ethanol is preferably chosen.

In another preferred embodiment, the drying in step d) is performed at a temperature of not more than 50° C. in a vacuum, preferably at not more than 40° C.

### EXAMPLES

### Example 1

### Synthesis of 4-nitro-oxy-methyl-benzoic acid (I)

a) Reaction of 4-chloromethyl-benzoic acid III with AgNO₃ and in the Presence of H₂SO₄
9.29 kg of 4-chloromethyl-benzoic acid (III) were added to 92.9 l of acetonitrile with stirring for 20 minutes, under a slow nitrogen current. 93 ml of sulphuric acid were added and the mixture was stirred for 15 minutes. 13.65 kg of silver nitrate were added, following the same operation conditions as when adding (III). The reactor was protected from direct exposure to light and the mixture was stirred for 15 minutes. The mixture was then refluxed for 7 hours and 15 minutes. The reaction mix was cooled down to 20° C.-25° C. 37.2 l of dimethylformamide were added and it was stirred for 30 minutes, keeping the temperature between 25° C. and 20° C.

### b) Separation of the Silver Salts by Filtration

The silver salts were separated by filtration, under nitrogen pressure, through a filter containing 9 kg of cellulose, previously washed with 111 l of water and three times with 28 l of dimethylformamide. The separated solid waste was washed twice with 9.3 l of dimethylformamide. The cellulose was withdrawn from the filter and washed with dimethylformamide until running clear and it was then rinsed with water.

c) Precipitation with Water

The liquid phases were put together and the temperature was stabilised to between 25° C. and 20° C. 1486 l of water were added for 1 hour, maintaining the temperature between 20° C. and 25° C. The mixture was stirred for 1 hour, maintaining the temperature between 20° C. and 25° C. The precipitate was separated by filtration, and the cake thus obtained was washed with water until obtaining a pH similar to that of the water. The cake was finally washed with 18.6 l of ethanol.

### d) Drying

The wet solid was dried at a temperature of not more than 40° C. in a vacuum until the KF water content was of 0.2% at the most. 9.68 kg of 4-nitro-oxy-methyl-benzoic acid (I) were obtained. Yield 90.2%. HPLC Purity 99.35%. Content of (IV) 0.23%.

1. A process for preparing 4-nitro-oxy-methyl-benzoic acid, with formula (I) 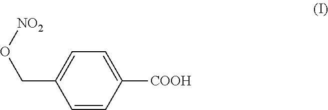 [SMILES: O=C(O)c1ccc(CO[N+](=O)[O-])cc1]
comprising the following steps:
a) reaction of 4-chloromethyl-benzoic acid (III) 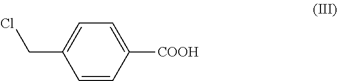 [SMILES: O=C(O)c1ccc(CCl)cc1]
with silver nitrate and in the presence of an acid as a catalyst in acetonitrile at reflux temperature, followed by cooling and adding of a polar aprotic solvent;
b) separation of the silver salts by filtration, followed by washout with a polar aprotic solvent;
c) precipitation of compound (I) with water from the filtrate of step b); and
d) drying of the compound (I).

2. The process according to claim 1, wherein the acid is chosen from the group consisting of benzene sulphonic, hydrobromic, hydrochloric, chloroacetic, chloro sulphonic, ethane sulphonic, phosphoric, methane sulphonic, nitric, p-chloro benzene sulphonic, p-toluene sulphonic, sulphuric, trichloroacetic, trichloromethane sulphonic, trifluoroacetic and trifluoromethane sulphonic and mixtures thereof.

3. The process according to claim 1, wherein the polar aprotic solvent in step a) is chosen form the group consisting of acetonitrile, benzonitrile, dimethylformamide, dimethyl sulphoxide, dioxane, N-methyl-2-pyrrolidone, propionitrile and tetrahydrofurane.

4. The process according to claim 1, wherein the polar aprotic solvent in step b) is chosen form the group consisting of acetonitrile, benzonitrile, dimethylformamide, dimethyl sulphoxide, dioxane, N-methyl-2-pyrrolidone, propionitrile and tetrahydrofurane.

5. The process according to claim 1, comprising in step c) a subsequent washout with (C₁-C₃)alkanol.

6. The process according to claim 1, wherein the drying in step d) is performed at a temperature of not more than 50° C. under vacuum.

7. The process according to claim 2, wherein the acid is sulphuric acid.

8. The process according to claim 3, wherein the polar aprotic solvent is dimethylformamide.

9. The process according to claim 4, wherein the polar aprotic solvent is dimethylformamide.

10. The process according to claim 5, wherein the (C₁-C₃)alkanol is ethanol.

11. The process according to claim 6, wherein the temperature is not more than 40° C.
